(19) United States
(12) Patent Application Publication
(10) Pub. No.: US 2024/0374140 A1
(43) Pub. Date: Nov. 14, 2024

(54) IMPLANTABLE MEDICAL DEVICES AND WIRELESS SENSOR ATTACHMENT

(71) Applicants: W. L. Gore & Associates, Inc., Newark, DE (US); Integrated Sensing Systems, Inc., Ypsilanti, MI (US)

(72) Inventors: David J. Minor, Phoenix, AZ (US); Edward E. Shaw, Flagstaff, AZ (US); Benjamin M. Trapp, Phoenix, AZ (US); Nader Najafi, Ann Arbor, MI (US)

(21) Appl. No.: 18/686,777

(22) PCT Filed: Aug. 25, 2022
(86) PCT No.: PCT/US2022/041457

§ 371 Date: Feb. 26, 2024

Related U.S. Application Data
(60) Provisional application No. 63/237,878, filed on Aug. 27, 2021.

(51) Int. Cl. A61B 5/00; A61B 5/0215

(52) U.S. Cl. CPC A61B 5/0031; A61B 5/0215; A61B 5/6869; A61B 5/6882; A61B 2562/12; A61B 2562/182

(57) ABSTRACT

Embodiments of the present disclosure relate to implantable medical devices configured to be implanted within a heart with anchor arrangements that reduce or eliminate signal transmission interference of the sensor. The implantable medical devices may include a sensor configured to sense physiological parameters of the heart and a housing comprising a transmitter and an antenna. An anchoring device formed by one or more wound wires including an engagement component is arranged around at least a portion of the housing a first frame component, a second frame component. The first frame component, the second frame component, or the first frame component and the second frame component comprise a first end separated from a second end to form a discontinuous loop and are formed from a conductive material.

### CROSS-REFERENCE TO RELATED APPLICATION

This application is a national phase application of PCT Application No. PCT/US2022/041457, internationally filed on Aug. 25, 2022, which claims the benefit of Provisional Application No. 63/237,878, filed Aug. 27, 2021, which are incorporated herein by reference in their entireties for all purposes.

### FIELD

The present disclosure relates generally to apparatuses, systems, and methods that include a sensing unit and an anchoring device for the sensing unit. More particularly, the apparatuses, systems, and methods are directed toward an implantable sensing unit to measure one or more physiological parameters of the heart and an anchoring device that reduces the likelihood of transmission interference of signals sent to and from the implantable sensing unit.

### BACKGROUND

Sensors may be implanted within a patient to acquire data. In certain instances, the sensor may be arranged or coupled to an implantable medical device.

### SUMMARY

According to one example (“Example 1”), an implantable medical device configured to be implanted within a heart that reduces or eliminates a likelihood of signal transmission interference that includes a sensor configured to sense physiological parameters of the heart; a housing comprising a transmitter and an antenna, wherein the sensor is coupled to the housing, and wherein the transmitter and the antenna are configured to transmit the sensed physiological parameters of the heart; and an anchoring device formed by one or more wires including an engagement component arranged around at least a portion of the housing; a first frame component configured to engage a first wall defining a first chamber of the heart; a second frame component configured to engage a second wall defining a second chamber of the heart; and the first frame component, the second frame component, or the first frame component and the second frame component comprise a first end separated from a second end to form a discontinuous loop and are formed from a conductive material.

According to one example (“Example 2”), the implantable medical device of Example 1, the discontinuous loop is arranged about the sensor at a position where an antenna of the sensor is located.

According to one example (“Example 3”), the implantable medical device of Example 1, at least a portion of the conductive material is covered in a non-conductive material.

According to one example (“Example 4”), the implantable medical device of any one of Examples 1-3, the first end, the second end, or the first end and the second end are laminated in a non-conductive material.

According to one example (“Example 5”), the implantable medical device of any one of Examples 3 or 4, wherein the non-conductive material is a membrane.

According to one example (“Example 6”), the implantable medical device of any one of Examples 1-5, the sensed physiological parameters comprise pressure measurements from at least one chamber of the heart.

According to one example (“Example 7”), the implantable medical device of any one of Examples 1-6, the engagement component includes a ring and the sensor defines a groove in cross-section.

According to one example (“Example 8”), the implantable medical device of any one of Examples 1-7, the one or more wires of the conductive material form one or more contact features in each of the first frame component and the second frame component

According to one example (“Example 9”), an anchoring device configured to be implanted within a heart that reduces or eliminates a likelihood of signal transmission interference that includes an engagement component configured to be arranged around at least a portion of a housing of an implantable sensor for the heart; a first frame component configured to engage a first wall defining a first chamber of the heart; a second frame component configured to engage a second wall defining a second chamber of the heart; and the first frame component, the second frame component, or the first frame component and the second frame component comprise a first end separated from a second end to form a discontinuous loop and are formed from a conductive material.

According to one example (“Example 10”), the anchoring device of Example 9, at least a portion of the conductive material is covered in a non-conductive material.

According to one example (“Example 11”), the anchoring device of any one of Examples 9 or 10, the first end, the second end, or the first end and the second end are laminated in a non-conductive material.

According to one example (“Example 12”), the anchoring device of any one of Examples 10 or 11, the non-conductive material is a membrane.

According to one example (“Example 13”), the anchoring device of any one of Examples 9-12, the sensed physiological parameters comprise pressure measurements from at least one chamber of the heart.

According to one example (“Example 14”), the anchoring device of any one of Examples 9-13, the engagement component has a reduced cross section that mates a groove in the sensor.

According to one example (“Example 15”), a method of manufacturing an implantable medical device that reduces or eliminates a likelihood of signal transmission interference, the method includes: receiving a housing comprising a transmitter, an antenna, and a sensor coupled to the housing, wherein the transmitter and the antenna are configured to transmit the sensed physiological parameters of the heart; and arranging an engagement component of an anchoring device at least partially around the housing, wherein the anchoring device comprises: a first frame component configured to engage a first wall defining a first chamber of the heart; a second frame component configured to engage a second wall defining a second chamber of the heart; and wherein the first frame component, the second frame component, or the first frame component and the second frame component comprise a first end separated from a second end to form a discontinuous loop and formed from a conductive material.

According to one example (“Example 16”), the method of Example 15, at least a portion of the conductive material is covered in a non-conductive material.

According to one example (“Example 17”), the method of Example 15, the anchoring device is formed of one or more wires.

According to one example (“Example 18”), an implantable medical device configured to be implanted within a heart that reduces or eliminates a likelihood of signal transmission interference that includes a sensor configured to sense physiological parameters of the heart; a housing comprising a transmitter and an antenna, wherein the sensor is coupled to the housing, and wherein the transmitter and the antenna are configured to transmit the sensed physiological parameters of the heart; and an anchoring device that includes an engagement component comprising a conductive material and arranged around at least a portion of the housing, the engagement component having a first end and a second end that are separated by a slit extending along a length of the engagement component radially separating a circumferential loop surrounding the portion of the housing; a first frame component configured to engage a first wall defining a first chamber of the heart; and a second frame component configured to engage a second wall defining a second chamber of the heart.

According to one example (“Example 19”), the implantable medical device of Example 18, the conductive material is a laser cut tube with a slit rendering it discontinuous.

According to one example (“Example 20”), the implantable medical device of Example 18, the engagement component includes a proximal portion, an intermediate portion, and a distal portion, the slit is arranged in the intermediate portion, and the proximal portion and the distal portion extend respectively beyond the first frame component and the second frame component.

According to another example (“Example 21”), a method of monitoring a physiologic parameter of the body with the implantable medical device of Example 18 includes monitoring the physiologic parameter in the first chamber of the heart with the sensor and monitoring the physiologic parameter in the second chamber of the heart with the sensor.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example implantable medical device for sensing blood pressure in accordance with at least one embodiment.

FIG. 2A is a side view of an example sensing unit and FIG. 2B is an axial view of the sensing unit shown in FIG. 2A in accordance with at least one embodiment.

FIG. 3 is a perspective view of an example anchoring device in accordance with at least one embodiment.

FIG. 4 is a perspective view of another example anchoring device in accordance with at least one embodiment.

FIG. 5 is a side view of an example sensing unit and anchoring device in accordance with at least one embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward arranging an implantable medical device including an implantable sensing unit and an anchoring device within a patient. The implantable medical device, as discussed herein, may minimize risk of embolization, minimize risk of creating loose thrombus, and/or minimize risk of enabling loose tissue growth-all of which can cause stroke or other clinical sequelae.

In certain instances, the implantable sensing unit may be arranged with and/or coupled to an anchoring device. The implantable sensing unit, which may be a wireless pressure sensor, may monitor heart failure and titrate medical therapy to prevent heart failure hospitalizations. In certain instances, the sensing unit may be arranged within the atrial septum of the heart and can access one or both left and right atrial pressures. Further, the sensing unit, as discussed herein, may protrude beyond the septal wall to avoid the tissue overgrowth over the sensor face (which may disturb the pressure reading). The anchoring device attached to the sensing unit as described herein reduces the likelihood of transmission interference of signals sent to and from the implantable sensing unit.

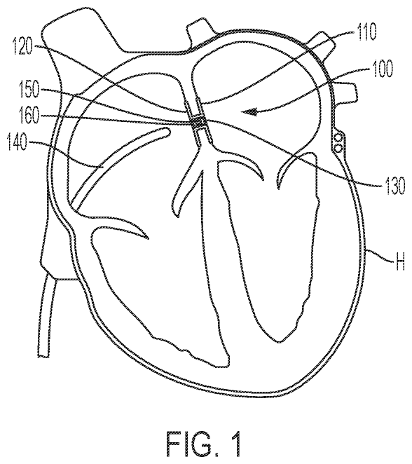

FIG. 1 is an example implantable medical device 100 for sensing blood pressure in accordance with an embodiment. This diagram is merely an example, which should not unduly limit the scope of the claims. One of ordinary skill in the art would recognize many variations, alternatives, and modifications.

The implantable medical device 100 is shown implanted within a heart H of a patient. The medical device 100 is shown arranged between the patient's left atrium and right atrium. In certain instances, the medical device 100 may be used to sense physiological parameters of the heart H and/or measure blood pressure or pressures within the heart H. For example, the medical device 100 may sense pressures of one or more chambers of the heart H, such as sense pressures in the left and right atriums LA, RA, and/or regulate blood flow within one or more chambers of the heart H, for example, between the left and right atria LA, RA. In certain embodiments, the medical device 100 includes a first frame component 110 arranged on a first side of a septum (e.g., within the right atrium RA), a second frame component 120 arranged on a second side of the septum (e.g., within the left atrium LA), and an engagement component 130 extending through the septum. A needle may be used to create an opening in the septum. According to certain embodiments, the first frame component 110, the second frame component 120, and the engagement component 130 may be collectively referred to herein as an anchoring device. In certain instances, the engagement component 130 includes a conical or cylindrical cross section.

In certain instances, the first and second frame components 110, 120 can be formed a single continuous wire or two separate wires. Additionally, or alternatively, the first and second frame components 110, 120 and/or the engagement component 130 can be formed from and/or include a shape-memory conductive material. For example, nitinol (NiTi) may be used as the material of the first and second frame components 110, 120 and/or the engagement component 130 (and any of the frame components and/or engagement components discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the first and second frame components 110, 120 and/or the engagement component 130. Because the first and second frame components 110, 120 and/or the engagement component 130 can be formed from a conductive material, the first and second frame components 110, 120 and/or the engagement component 130 have the potential to interfere with signals transmitted to and from the sensing unit 150. According to certain embodiments, the first frame component 110, the second frame component 120, and/or the engagement component 130 can be configured to reduce or eliminate the likelihood of signal transmission and/or signal reception by the medical device 100, as described in more detail below.

A sheath 140 and constraining and/or release lines (not shown) may be used to facilitate deployment of the medical device 100. For example, a first side of the medical device 100 that includes the first frame component 110 may be released after the sheath 140 is advanced through the septum and to the LA, and the second frame component 120 that includes the second frame component 120 may be released on the RA side of the septum. The engagement component 130 is arranged within the opening. According to certain embodiments, the engagement component 130 can be incorporated into and/or coupled to one or both of the first and/or second frame components 110, 120. The first and second frame components 110, 120 and/or the engagement component 130 may be compressed within the sheath 140 during delivery of the medical device 100 to the desired treatment area within the patient and subsequently expanded during deployment of the medical device 100. In other instances, the implantable medical device 100 may be implanted at a different location in the heart or a different location within the patient (e.g., gastrointestinal system, vasculature, brain).

In certain instances, the engagement component 130 is configured to engage and/or couple to a sensing unit 150. The engagement component 130, in certain instances, may extend beyond each of the first frame component 110 and the second frame component 120 and/or between the first frame component 110 and the second first frame component 120 to facilitate positioning of the sensing unit 150. According to certain embodiments, the sensing unit 150 includes one or more electrical components 160 for sensing one or more physiological parameters within the heart H, for example, pressures of the left and/or right atriums LA, RA. Example electrical components 160 include, but are not limited to, one or more sensors, a processing unit for processing sensor measurements from the one or more sensors, memory to store one or more sensor measurements and/or store one or more processed signals, a transmitter/receiver, and/or an antenna for transmitting one or more sensor measurements to another device and/or receiving one or more signals from another device. To reduce the likelihood of signal interference of one or more signals sent and/or received by the electrical components 160, the first frame component 110, the second frame component 120, and/or the engagement component 130 can form a discontinuous loop around the sensing unit 150 and/or antenna, as explained in more detail below.

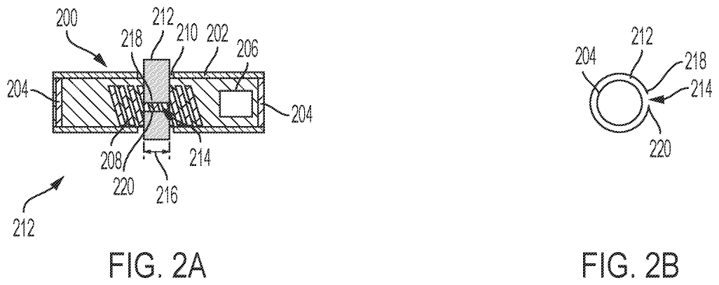

FIG. 2A is a side view of an example sensing unit 200 and FIG. 2B is an axial view of the sensing unit 200 shown in FIG. 2A in accordance with at least one embodiment. These diagrams are merely examples, which should not unduly limit the scope of the claims. One of ordinary skill in the art would recognize many variations, alternatives, and modifications.

The sensing unit 200 can be the same or similar to the sensing unit 150 described above in relation to FIG. 1. For example, the sensing unit 200 can be configured to sense one or more physiological parameters within the heart H, for example, the pressures of the left and/or right atriums LA, RA. According to certain embodiments, the sensing unit 200 can be coupled to one or more frame components (e.g., first and second frame components 110, 120, 302, 304, 404, 406) and/or an engagement component (e.g., engagement component 130, 212, 306, 408) and placed within the heart H, for example, within the atrial septum. As discussed above in relation to FIG. 1, one or more frame components and/or an engagement component coupled to the sensing unit 200 can secure the sensing unit 200 to a location within the heart H (e.g., the atrial septum).

According to certain embodiments, the sensing unit 200 includes a sensor housing 202. In some instances, an engagement component 212 can be configured to engage the sensor housing 202 in order to couple one or more of the first and second frame components 110, 120 to the sensor housing 202. In certain instances, the engagement component 212 includes a ring or groove cross section. For example, in some instances, the engagement component 212 includes a reduced cross section that mates a groove in the sensor.

In certain instances, the sensor housing 202 can be formed from a non-conductive material, e.g., glass or ceramic. Additionally, or alternatively, the sensor housing 202 can be formed from and/or coated in a biocompatible material. For example, the sensor housing 202 can be coated in a fluoropolymer, such as a polytetrafluoroethylene (PTFE) polymer or an expanded polytetrafluoroethylene (ePTFE) polymer. In some instances, the sensor housing 202 may be formed and/or include, but not limited to, one or more of the following materials: a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the sensor housing 202 can include Dacron, polyolefins, carboxy methylcellulose fabrics, polyurethanes, or other woven, non-woven, or film elastomers.

According to certain embodiments, a sensing element 204 is arranged at one or more both ends of the sensing unit 200. The sensing element 204 can be configured to sense one or more physiological parameters in the heart H, for example, pressures in the left and/or right atriums LA, RA.

In certain instances, the sensed physiological parameters by the sensing element 204 are transmitted to one or more electrical components 206 arranged within the sensor housing 202. Example electrical components 206 include, but are not limited to a processing unit, memory, a power supply, a transmitter, and/or a receiver. In certain instances, the processing unit is configured to process one or more sensed physiological parameters from the sensing element 204 (e.g., convert an analog sensor measurements to a digital signal, reduce noise, and/or perform other signal processing on the sensor measurements). The memory can be configured to store one or more sensed physiological parameters from the sensing element 204 and/or store signals processed by the processing unit. The transmitter can be configured to wirelessly transmit, via an antenna 208, one or more sensor measurements from sensing element 204 to another device. The receiver can be configured to receive signals from another device. And, the power supply can be configured to supply power to the sensing element 204 and/or one or more of the electrical components 206.

In some embodiments, the sensing unit 200 includes a notch 210. While the illustrated embodiment shows one notch 210, in certain instances, the sensing unit 200 includes more than one notch 210. The notch(s) 210 can be an indentation and/or a cutout of the sensor housing 202. In some instances, the notch(s) 210 facilitates coupling the engagement component 212 and/or one or more frame components to the sensing unit 200. For example, the notch(s) 210 can reduce the likelihood that the engagement component 212 and/or one or more frame components translate toward one side of the sensing unit 200 or the other.

Due to the sensing unit 200 being configured to wirelessly transmit one or more sensed physiological parameters from the sensing unit 200 to another device and/or wirelessly receive one or more signals from another device, it is important for interference of the signals to be reduced. Conventional frame components oftentimes can interfere with said signals because they form a continuous conductive loop around a sensing unit (including the antenna). One or more frame components and/or the engagement components disclosed herein, however, are designed to reduce the likelihood of signal interference, as described in more detail below.

According to certain embodiments, the engagement component 212 is coupled to the sensing unit 200. In certain instances, the engagement component 212 is coupled to and/or formed with the first and second frame components 110, 120. In certain instances, the engagement component 212 can at least partially surround the sensor housing 202, e.g., the notch 210.

According to some embodiments, the engagement component 212 can be formed from and/or include a shape-memory conductive material. For example, NiTi may be used as the material of the engagement component 212 (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the engagement component 212. The super-elastic properties and softness of NiTi may enhance the conformability of the engagement component 212. In addition, NiTi can be shape-set into a desired shape. That is, NiTi can be shape-set so that the frame tends to self-expand into a desired shape when the engagement component 212 is unconstrained, such as when the engagement component 212 is deployed out from a delivery system.

Because the engagement component 212 can be formed from a conductive material, the engagement component 212 has the potential to interfere with signals transmitted to and from the sensing unit 200. To reduce the likelihood of signal interference, the engagement component 212 can include a slit 214. In certain instances, the slit 214 may be referred to herein as a gap, a discontinuity, and/or the like. In instances, the slit 214 extends along an entire length 216 of the engagement component 212. For example, the engagement component 212 can include a first end 218 that is separated from a second end 220 by a space 214, such as the slit 214. In instances, the space 214 can be air and/or can be filled with a dielectric, such as glass, plastic, etc. Additionally, or alternatively, the engagement component 212 can be coated in a non-conductive material, such as a fluoropolymer, such as a PTFE polymer or an ePTFE polymer.

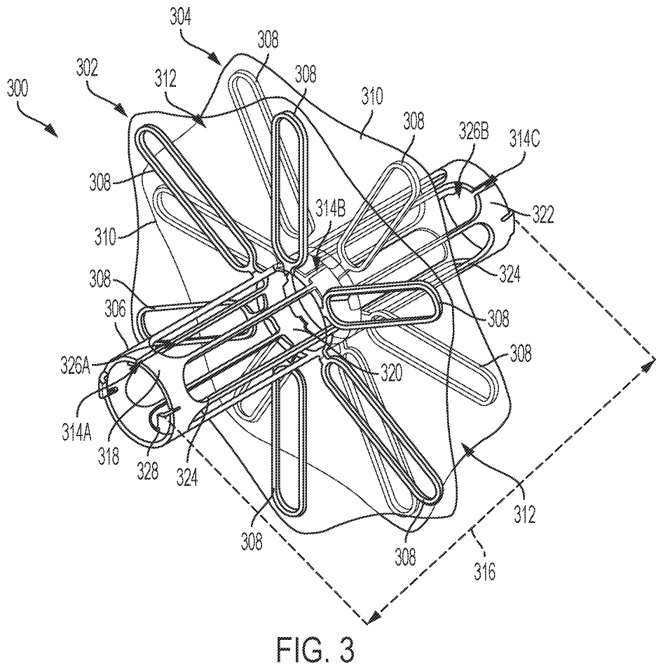

FIG. 3 is a perspective view of an example anchoring device 300, according to at least one embodiment. This diagram is merely an example, which should not unduly limit the scope of the claims. One of ordinary skill in the art would recognize many variations, alternatives, and modifications.

According to certain embodiments, the anchoring device 300 can be coupled to a sensing unit 150, 200 in order to position the sensing unit 150, 200 within a heart H, for example, within the atrial septum of the heart H.

According to certain embodiments, the anchoring device 300 includes a first frame component 302, a second frame component 304, and an engagement component 306. The first frame component 302 and the second frame component 304 can have the same or similar characteristics as the first frame component 110 and the second frame component 120, respectively. For example, the first frame component 302 may be arranged on a first side of an atrial septum and the second frame component 304 may be arranged on a second side of the atrial septum to secure the anchoring device 300 and the sensing unit 150, 200 within the heart H. As another example, the first and second frame components 302, 304 can be formed a single continuous wire or two separate wires. Additionally, or alternatively, the engagement component 306 can have the same or similar characteristics as the engagement component 212 discussed above in relation to FIG. 2. In certain instances, the anchoring device 300 may be formed of a laser-cut tube.

According to certain embodiments, each of the first frame component 302 and the second frame component 304 can include one or more contact features 308 extending radially from the engagement component 306. In some instances, the contact features 308 have an ovular shape as shown. However, this is merely an example and not meant to be limiting; it is also contemplated that the contact features 308 can have other non-ovular shapes. In certain embodiments, the contact features 308 form a frame for the first frame component 302 and the second frame component 304, which are used to secure the anchoring component 300 within a heart H. In instances, the contact features 308 and/or the engagement component 306 can be formed from NiTi or another shape-memory material.

According to certain embodiments, the contact features 308 can be covered in a membrane 310. The membrane 310 may be arranged across the contact features 308 of the first frame component 302 and the contact features 308 of the second frame component 304. As shown, the membrane 310 spans areas 312 of the first frame component 302 and the second frame component 302 that do not include a contact feature 308 that forms the first frame component 302 and the second frame component 304. In certain instances, the membrane 310 may be formed from a non-conductive, biocompatible material such as a fluoropolymer, such as a PTFE polymer or an ePTFE polymer.

In certain instances, the contact features 308 are connected to engagement component 306 via the material used to form the contact features 308, as illustrated. Alternatively, the contact features 308 are connected to the engagement component 306 via the membrane 310.

According to certain embodiments, the engagement component 306 includes a slit 314, so that the engagement portion 306 does form a continuous conductor surrounding a sensing unit 150, 200. In certain instances, the slit 314 can have the same or similar characteristics as the slit 214 described above in relation to FIG. 2. For example, the slit 314 can extend along an entire length 316 of the anchoring device 300. Additionally, or alternatively, air and/or a dielectric, such as glass, plastic, etc. can be in between the slit 314. Additionally, or alternatively, the engagement component 306 can be coated in a non-conductive material, such as a fluoropolymer, such as a PTFE polymer or an ePTFE polymer. As described above, the slit 314 can reduce the likelihood the anchoring device 300 interferes with signals transmitted to and from a sensing unit 150, 200. As noted above, the anchoring device 300 may be a laser-cut tube and the slit 314 may be a cut portion of the laser-cut tube. The slit 314 in the laser-cut tube may render the anchoring device 300 discontinues.

In some embodiments, the engagement portion 306 includes a proximal portion 318, an intermediate portion 320, and/or a distal portion 322. The proximal portion 318 can be connected to the intermediate portion 320 by one or more struts 324. Additionally, or alternatively, the intermediate portion 320 can be connected to the distal portion 322 by one or more struts 324. In some embodiments, the struts 324 connect the portions 318, 320, 322 as well as space apart the portions 318, 320, 322 from one another. As illustrated, the struts 324 can include gaps 326 therebetween. The proximal portion 318 and the distal portion 322 may extend beyond the first frame component 302 and the second frame component 304. In certain instances, the slit 314 extends along a length of the engagement component 306 radially separating a circumferential loop surrounding a portion of the housing of the sensing unit (e.g., where the antenna is located).

In at least some embodiments, one or more slits 314 connect with one or more gaps 326 to prevent the anchoring device 300 from forming a continuous conductor around a sensing unit (e.g., sensing unit 150, 200). For example, as illustrated, the slit 314A connects to the gap 326A. The gap 326A in turn connects to the slit 314B. The slit in turn connects to the gap 326B and the gap 326B connects to the slit 314C. Because slits 314 and the one or more gaps 326 do not form a continuous conductor around a sensing unit, the anchoring device 300 is less likely to interfere with transmission sent from and received by the sensing unit. The intermediate portion 320 may be circumferentially continuous about the sensing unit with the exception of one or more slits 314.

According to certain embodiments, the engagement component 306 includes one or more bosses 328 extending radially inward from the proximal portion 318 and/or radially inward from the distal portion 322. In certain instances, the bosses 328 can facilitate centering and/or coupling of the anchoring device 300 to a sensing unit.

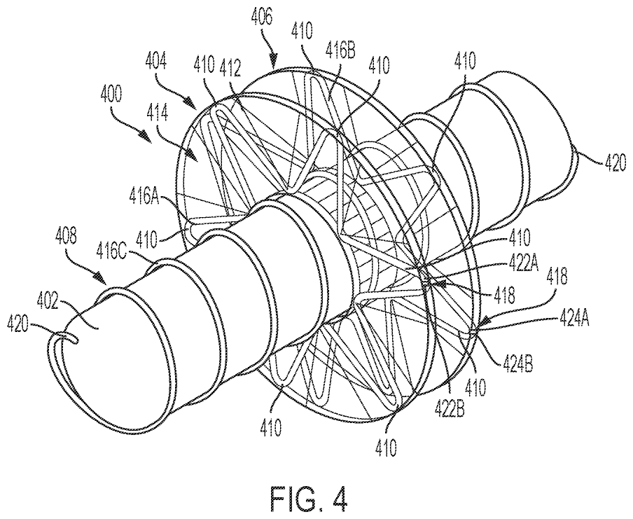

FIG. 4 is a perspective view of another example anchoring device 400 in accordance with at least one embodiment. This diagram is merely an example, which should not unduly limit the scope of the claims. One of ordinary skill in the art would recognize many variations, alternatives, and modifications.

According to certain embodiments, the anchoring device 400 can be coupled to a sensing unit 402 in order to position the sensing unit 402 within a heart H, for example, within the atrial septum of the heart H. In certain embodiments, the sensing unit 402 can have the same or similar characteristics as the sensing unit 150, 200 describe above.

According to certain embodiments, the anchoring device 400 includes a first frame component 404, a second frame component 406, and an engagement component 408. The first frame component 404 and the second frame component 406 can have the same or similar characteristics as the first frame components 110, 302 and/or the second frame components 120, 304, respectively. For example, the first frame component 404 may be arranged on a first side of an atrial septum and the second frame component 406 may be arranged on a second side of the atrial septum to secure the anchoring device 400 and the sensing unit 402 within the heart H. As another example, the first and second frame components 404, 406 can be formed a single continuous wire or two separate wires. Additionally, or alternatively, the engagement component 408 can have the same or similar characteristics as the engagement components 212, 306.

According to certain embodiments, each of the first and second frame components 404, 406 can include one or more contact features 410 extending radially from the engagement component 408. In some instances, the contact features 410 have a star shape as shown. However, this is merely an example and not meant to be limiting; it is also contemplated that the contact features 410 can have other non-star shapes. In certain embodiments, the contact features 410 form a frame for the first frame component 404 and the second frame component 406, which are used to secure the anchoring device 400 within a heart H. In instances, the contact features 410 and/or the engagement component 408 can be formed from NiTi or another shape-memory material.

According to certain embodiments, the contact features 410 can be covered in a membrane 412. The membrane 412 may be arranged across the contact features 410 of the first frame component 404 and the contact features 410 of the second frame component 406. As shown, the membrane 412 spans areas 414 of the first frame component 404 and the second frame component 406 that do not include a contact feature 410 that forms the first frame component 404 and the second frame component 406. In certain instances, the membrane 412 may be formed from a non-conductive, biocompatible material such as a fluoropolymer, such as a PTFE polymer or an ePTFE polymer.

According to certain embodiments, the engagement component 408 and the contact features 410 can be formed from one or more wires 416. For example, the first frame component 404 can be formed using a wire 416A, the second frame component 406 can be formed using a wire 416B, and/or the engagement component 408 can be formed using a wire 416C. In some embodiments, the wires 416A, 416B, 416C are separate wires or the same wire.

In certain embodiments, each of the wires 416A, 416B used to form the first and second frame components 404, 406 include a discontinuity 418 so neither of the wires 416A, 416B for the first and second frame components 404, 406 continuously surround the sensing unit 402. Additionally, or alternatively, the wire 416C used to form the engagement component 408 does not form a continuous loop around the sensing unit 402. For example, the ends 420 of wire 416C do not connect and, therefore, do not form a continuous conductor around the sensing unit 402. Because none of the wires 416A, 416B, 416C form a continuous conductor around the sensing unit 402, the wires 416A, 416B, 416C are less likely to interfere with transmission sent from and received by the sensing unit 402. In certain instances, the wires 416A, 416B, 416C, individually or taken collectively, do not form a conductive loop about the sensing unit 402

In certain embodiments, to reduce the likelihood the one or more wires 416 forming a continuous conductor around the sensing unit 402 by, for example, different portions of the one or more wires 416 coming into contact with one another to form a continuous conductor around the sensing unit 402, the one or more wires 416 can be coated (e.g., laminated) with a non-conductive material extending along an entire length of the one or more wires 416 or a portion of the length of the one or more wires 416. Additionally, or alternatively, to reduce the likelihood the one or more wires 416 forming a continuous conductor around the sensing unit 402, a sleeve and/or a jacket can be arranged over an entire length of the one or more wires 416 or a portion of the length of the one or more wires 416. In certain embodiments, the non-conductive material is a fluoropolymer, such as a PTFE polymer or an ePTFE polymer. In certain instances, an oxide layer of one or more wires 416 may form the non-conductive material. Additionally, or alternatively, the ends 422 of the first frame component 404 and/or the ends 424 of the second frame component 406 can be coated (e.g., laminated) with a non-conductive material, such as a fluoropolymer to reduce the likelihood of the wires 416A, 416B forming a continuous conductor around the sensing unit 402.

According to certain embodiments, the ends 420 can be curved over the ends of the sensing unit 402 to facilitate centering and/or coupling of the anchoring device 400 to the sensing unit 402.

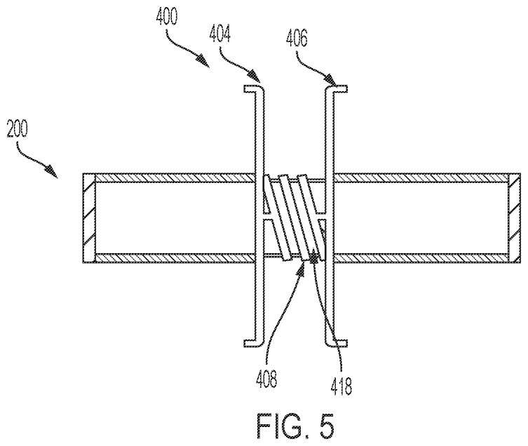

FIG. 5 is a side view of an example sensing unit 200 and anchoring device in accordance with at least one embodiment. This diagram is merely an example, which should not unduly limit the scope of the claims. One of ordinary skill in the art would recognize many variations, alternatives, and modifications.

According to certain embodiments, the anchoring device 400 can be coupled to a sensing unit 402 in order to position the sensing unit 402 within a heart H, for example, within the atrial septum of the heart H. In certain embodiments, the sensing unit 402 can have the same or similar characteristics as the sensing unit 150, 200 describe above.

According to certain embodiments, the anchoring device 400 includes a first frame component 404, a second frame component 406, and an engagement component 408. The first frame component 404 and the second frame component 406 can have the same or similar characteristics as the first frame components 110, 302 and/or the second frame components 120, 304, respectively. For example, the first frame component 404 may be arranged on a first side of an atrial septum and the second frame component 406 may be arranged on a second side of the atrial septum to secure the anchoring device 400 and the sensing unit 402 within the heart H. As another example, the first and second frame components 404, 406 can be formed a single continuous wire or two separate wires. Additionally, or alternatively, the engagement component 408 can have the same or similar characteristics as the engagement components 212, 306.

In certain instances, the first and second frame components 110, 120 can be formed a single continuous wire or two separate wires. As shown in FIG. 5, the engagement component 408 may be formed of the one or more wires. The engagement component 408 may be arranged about the sensing unit 200 (in certain instances in a section of the sensing unit 200 where the antenna is located). The engagement component 408 may include a discontinuity 418 so neither of the wires of the first and second frame components 404, 406 continuously surround the sensing unit 402.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An implantable medical device configured to be implanted within a heart that reduces or eliminates a likelihood of signal transmission interference, the implantable medical device comprising:
a sensor configured to sense physiological parameters of the heart;
a housing comprising a transmitter and an antenna, wherein the sensor is coupled to the housing, and wherein the transmitter and the antenna are configured to transmit the sensed physiological parameters of the heart; and
an anchoring device formed by one or more wires comprising:
an engagement component arranged around at least a portion of the housing;
a first frame component configured to engage a first wall defining a first chamber of the heart;
a second frame component configured to engage a second wall defining a second chamber of the heart; and
wherein the first frame component, the second frame component, or the first frame component and the second frame component comprise a first end separated from a second end to form a discontinuous loop and are formed from a conductive material.

2. The implantable medical device of claim 1, wherein the discontinuous loop is arranged about the sensor at a position where an antenna of the sensor is located.

3. The implantable medical device of claim 1, wherein at least a portion of the conductive material is covered in a non-conductive material.

4. The implantable medical device of claim 1, wherein the first end, the second end, or the first end and the second end are laminated in a non-conductive material.

5. The implantable medical device of claim 3, wherein the non-conductive material is a membrane.

6. The implantable medical device of claim 1, wherein the sensed physiological parameters comprise pressure measurements from at least one chamber of the heart.

7. The implantable medical device of claim 1, wherein the engagement component includes a ring and the sensor defines a groove in cross-section.

8. The implantable medical device of claim 1, wherein the one or more wires of the conductive material form one or more contact features in each of the first frame component and the second frame component.

9. An anchoring device configured to be implanted within a heart that reduces or eliminates a likelihood of signal transmission interference, the implantable device comprising one or more wound wires forming:
an engagement component configured to be arranged around at least a portion of a housing of an implantable sensor for the heart;
a first frame component configured to engage a first wall defining a first chamber of the heart;
a second frame component configured to engage a second wall defining a second chamber of the heart; and
wherein the first frame component, the second frame component, or the first frame component and the second frame component comprise a first end separated from a second end to form a discontinuous loop and are formed from a conductive material.

10. The anchoring device of claim 9, wherein at least a portion of the conductive material is covered in a non-conductive material.

11. The anchoring device of claim 9, wherein the first end, the second end, or the first end and the second end are laminated in a non-conductive material.

12. The anchoring device of claim 10, wherein the non-conductive material is a membrane.

13. The anchoring device of claim 9, wherein the sensed physiological parameters comprise pressure measurements from at least one chamber of the heart.

14. The anchoring device of claim 9, wherein the engagement component has a reduced cross section that mates a groove in the sensor.

15. A method of manufacturing an implantable medical device that reduces or eliminates a likelihood of signal transmission interference, the method comprising:
receiving a housing comprising a transmitter, an antenna, and a sensor coupled to the housing, wherein the transmitter and the antenna are configured to transmit the sensed physiological parameters of the heart; and
arranging an engagement component of an anchoring device at least partially around the housing, wherein the anchoring device comprises:
a first frame component configured to engage a first wall defining a first chamber of the heart;
a second frame component configured to engage a second wall defining a second chamber of the heart; and
wherein the first frame component, the second frame component, or the first frame component and the second frame component comprise a first end separated from a second end to form a discontinuous loop and formed from a conductive material.

16. The method of claim 15, wherein at least a portion of the conductive material is covered in a non-conductive material.

17. The method of claim 15, wherein the anchoring device is formed of one or more wires.

18. An implantable medical device configured to be implanted within a heart that reduces or eliminates a likelihood of signal transmission interference, the implantable medical device comprising:
a sensor configured to sense physiological parameters of the heart;
a housing comprising a transmitter and an antenna, wherein the sensor is coupled to the housing, and wherein the transmitter and the antenna are configured to transmit the sensed physiological parameters of the heart; and
an anchoring device comprising:
an engagement component comprising a conductive material and arranged around at least a portion of the housing, the engagement component having a first end and a second end that are separated by a slit extending along a length of the engagement component radially separating a circumferential loop surrounding the portion of the housing;
a first frame component configured to engage a first wall defining a first chamber of the heart; and
a second frame component configured to engage a second wall defining a second chamber of the heart.

19. The implantable medical device of claim 18, wherein the conductive material is a laser cut tube with a slit rendering it discontinuous.

20. The implantable medical device of claim 18, wherein the engagement component includes a proximal portion, an intermediate portion, and a distal portion, the slit is arranged in the intermediate portion, and the proximal portion and the distal portion extend respectively beyond the first frame component and the second frame component.

21. A method of monitoring a physiological parameter of the body with the implantable medical device of claim 18, the method including monitoring the physiological parameter in the first chamber of the heart with the sensor and monitoring the physiological parameter in the second chamber of the heart with the sensor.
